(19) United States
(12) Patent Application Publication
(10) Pub. No.: US 2009/0162312 A1
(43) Pub. Date: Jun. 25, 2009

(54) NOVEL BIODEGRADABLE BIOFOULING CONTROL COATING AND METHOD OF FORMULATION

(76) Inventors: Ronald R. Price, Stevensville, MD (US); Eric R. Welsh, Annapolis, MD (US); Linda Passaro, New Fairfield, CT (US)

Correspondence Address: NAVAL RESEARCH LABORATORY;ASSOCIATE COUNSEL (PATENTS), CODE 1008.2, 4555 OVERLOOK AVENUE, S.W., WASHINGTON, DC, 20375-5320, US

(21) Appl. No.: 12/395,816

(22) Filed: Mar. 2, 2009

Related U.S. Application Data
(63) Continuation of application No. 11/229,433, filed on Sep. 15, 2005.

(51) Int. Cl. A61K 31/74

(52) U.S. Cl. 424/78.09

(57) ABSTRACT

An antifouling coating including a polymer made by crosslinking a chitosan with a blocked polyisocyanate.

This application is a continuation application of U.S. application Ser. No. 11/229,433, filed on Sep. 15, 2005, pending. The parent application is incorporated herein by reference.

### BACKGROUND OF THE INVENTION

1. Field of the Invention

The invention relates generally to antifouling coatings.

2. Description of the Related Art

Marine biofouling of submerged surfaces is difficult to control. Prior efforts to control marine biofouling include coating the surface with a protectant material such as copper sheeting, copper-nickel sheeting, copper containing paint formulations as well as tri(organotin) formulations, arsenicals, mecurials, and other toxic biocides. These formulations control biofouling well but tend to pollute the marine environment and often have a major effect on non-target species.

Other methods to control fouling such as cleanable coatings containing Teflon or PDMS silicones have proven to be less durable than desired and in addition suffer from adhesion problems and have proved to be less than stellar performers in the market place. Other methods that utilize electrical currents or sound emission have likewise proven to be difficult to install and maintain with limited success.

In order to reduce or eliminate the need for metallic biocides several non-metallic biocides have been introduced to the marketplace and have achieved environmental approval; however, they are difficult to formulate into a coating due to the fact that the base paint typically was designed for the release of copper ion into solution and are over plasticized by the small hydrophobic molecules. In some cases these commercial biocides have proven to target desirable marine species such as corals.

Natural product antifouling agents have been identified in the past as occurring from numerous marine species such as algae, corals, and sponges. Often these compounds are highly effective; however, they have been difficult to utilize in coatings formulations for a number of reasons, or have proven difficult to make by synthetic means. In addition many are easily degraded and thus do not survive well once mixed into a coating matrix. Despite these limitations several products have proven to be interesting and are the subject of patent applications and research. Several of these compounds are known to function well as antifoulants and either function as plasticizers in standard paint formulations or tend to be highly volatile and thus do not persist over time in the coating providing only limited lifetime performance.

Some marine organisms have mechanisms used to defend themselves from fouling. Initially there are species that utilize desquamation and similar surface strategies for the prevention of fouling and typically these are free-swimming large marine mammals where the speed through the water and the constant renewal of the skin prevents long term fouling of their surface. A self-polishing or ablative surface mimics this means of fouling prevention; however, unlike marine species that are capable of renewal of their surfaces on a constant basis, the paint formulator must be able to apply a slowly ablating surface sufficient to last the engineering goals for service life for the coating. Thus if constantly polishing, the lifetime would be fairly short unless multiple coats are utilized.

In the case of more non-motile species such as corals, sponges, and marine algae these species provide for defense by means that will repel a range of fouling and permit a rather fouling free surface over their lifetimes. In order to achieve this goal they have evolved the means of producing secondary metabolites that serve as toxic antifouling compounds or are active in repelling other fouling species by non-toxic means. Such compounds are the focus of interest for many researchers in the marine coatings field. One of the attractive points about marine chemical ecology is that for the most part the active agents are produced as a byproduct of the biochemistry of the plant or animal, and the products are readily degraded in the marine environment. Most of the compounds have poor water solubility, are small hydrophobic molecules, and are often produced in relation to the production and secretion of muco-polysaccharides onto the surface of the plant or animal that produces them. Thus many of the compounds are surface active rather than solution active and this would be expected, as the vast mass of seawater would rapidly dilute any agent that was readily soluble and solution active. If the compounds are surface active then the coating should be optimized to contain the maximum amount of the active agent possible, should be capable of presenting the compound to the coating surface and then provide a mechanism to renew that surface when the active agent is depleted.

Current coatings may slowly ablate by a physicochemical mode of action. Self-polishing paints mimic the activity of marine mammals by constantly eroding the outer surface exposing fresh paint and preventing marine fouling from sticking. These coatings do not address the need of having the maximum polishing rate occur only in times of high fouling settlement pressure. Thus a method of allowing the coating to degrade under fouling settlement pressure is needed.

### SUMMARY OF THE INVENTION

The invention comprises a polymer made by crosslinking a chitosan with a blocked polyisocyanate.

The invention further comprises a method of making a polymer comprising: providing an aqueous solution of a chitosan, and crosslinking the chitosan with a blocked polyisocyanate.

### BRIEF DESCRIPTION OF THE DRAWINGS

A more complete appreciation of the invention will be readily obtained by reference to the following Description of the Example Embodiments and the accompanying drawings.

FIG. 1 illustrates a process by which a coating of the polymer may act as an antifoulant.

### DETAILED DESCRIPTION OF EXAMPLE EMBODIMENTS

In the following description, for purposes of explanation and not limitation, specific details are set forth in order to provide a thorough understanding of the present invention. However, it will be apparent to one skilled in the art that the present invention may be practiced in other embodiments that depart from these specific details. In other instances, detailed descriptions of well-known methods and devices are omitted so as to not obscure the description of the present invention with unnecessary detail.

The polymer and coating being described herein is made from chitosan, which is a derivative of chitin, the second most abundant biological polymer on earth. Chitosan is of low toxicity, biocompatible and biodegradable, and offers a challenge to the coatings formulator, as it is only soluble at lower pH ranges in dilute organic acid solutions. This may make it difficult to process and thus a means is needed to crosslink the coating in a water-based environment. To this end, water dispersible polyfunctional or difunctional isocyanate crosslinkers are utilized to form the crosslinked coating in solution. To prevent a brittle coating a polyethylene glycol may be utilized to crosslink into the system increasing flexibility. This coating may be utilized to form a gel matrix or to form a hard polymeric film suitable for use as a marine coating. The chitothane is biodegradable by chitinase enzymes, lysozyme, and other enzymes and the rate of degradation is controlled by the degree of crosslinking of the chitosan matrix.

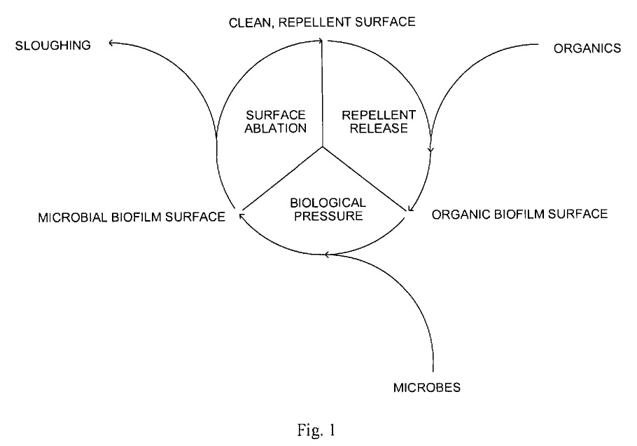

Thus with a biodegradable coating the coating will by definition renew the surface by polishing when the coating is under pressure from marine organisms that produce chitinase and other similar enzymes. FIG. 1 illustrates a cyclic process of biological pressure, surface ablation, and repellent release.

This coating is water based and depending on the crosslinking system may be utilized as a two component system or as a single package. The two part package is based on COTS soluble diisocyanate chemistry (Bayhydur 302 (Bayer)) and the one part system is based on a sulfonate-blocked methylene-diisocyanate. The crosslinking is facilitated by either an increase in pH or by heat. Being water based the coaling does not contain large quantities of volatile organic solvents and as it is crosslinked does not rely on coalescent solvents as do latex coatings.

The structure of chitosan is shown below in Eq. (1). When the isocyanate crosslinks the chitosan, both urea (Eq. (2)) and urethane (Eq. (3)) linkages may be formed, including combinations occurring from a single polyisocyanate molecule. Urea linkages may be preferentially formed.

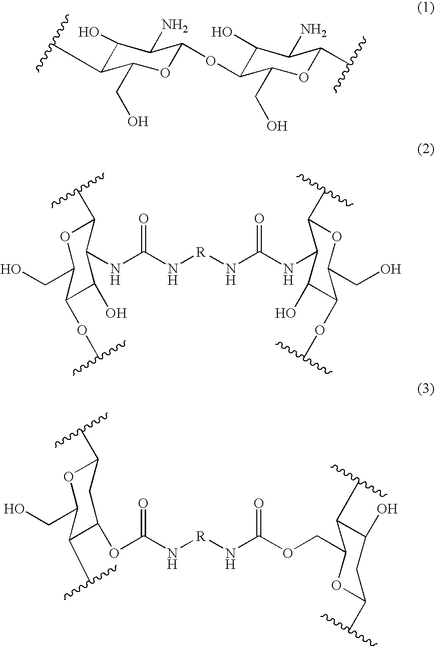

Suitable isocyanates include, but are not limited to, sulphone blocked hexamethylene diisocyanate and Bayhydur 302 blocked isocyanate. A sample structure of a polymer made from the former is shown in Eq. (4). Blocked isocyanates are used to prevent the product from foaming during crosslinking.

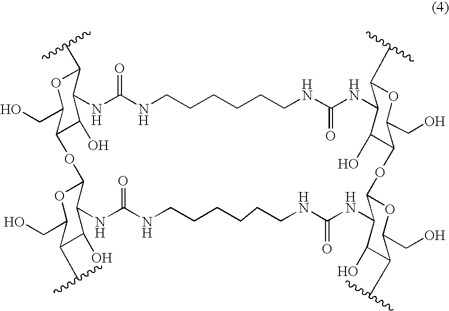

The crosslinking may also be performed by also crosslinking with a polyethylene glycol (PEG). An example general structure is shown in Eq. (5), The PEG has the effect of lengthening the crosslink between chitosan chains. The crosslink may have a single PEG repeat unit, as when m is 1. When m is higher, the crosslink contains multiple PEG groups and additional isocyanate residues.

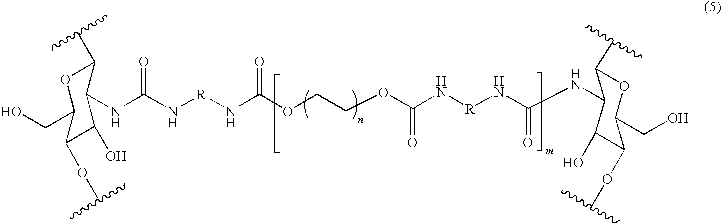

A coating comprising the polymer, such as an antifouling coating may be made by applying a mixture of the chitosan solution and the blocked polyisocyanate to a surface before the crosslinking.

The coating may have more effective antifouling properties when it further comprises a delivery system. Halloysite (Al₂Si₂O₅(OH)₄) clay in the form of microtubules, which is a naturally occurring material, is the basic chemical constituents of bentonite class clays commonly utilized in marine coatings and does not pose an environmental risk. The tubules are modified by entrapment of a biologically active compound inside the microtubules. Suitable biologically active compounds include, but are not limited to, a 2-furyl-alkylketone, a 2-furyl-pentylketone, a 2-furyl-heptylketone, a 2-furyl-octylketone, and a 2-furyl-nonylketone. The alkyl groups in these compounds may be substituted alkyl groups. The compound may remain effective for a longer period when the microtubules are coated with a polyethylene glycol polyurethane.

The 7-9 carbon analogs of 2-furyl-n-pentylketone (2-FNPK) are far less volatile then 2-FNPK and are bacteriostatic against many common strains of bacteria. The compound is a synthetic analog of furangemmecrene a known potent antifouling natural product. The mode of activity is non-toxic. This compound may be further modified by adding a functional group to the end of the acyl chain to allow the molecule to be bound at a surface.

The final coating may comprise the chitothane base as a two part component in water. The initial part is comprised of the active agent that has been entrapped in halloysite clay at a ratio of, for example, between 4 and 32% by weight.

Other components may also be added to the coating to potentially increase its effectiveness. Very high molecular weight polydimethylsiloxane (PDMS) solutions may be added in quantities such as between 0.5 and 1.0% by wt to the coating prior to the cross linking agent. This addition provides a means of making the surface hydrophobic in order to provide an environment wherein the hydrophobic active agent is capable migrating from the controlled delivery system and remaining.

An anti oxidant and UV inhibitor may also be added. The utilization of these stabilizing compounds allows for the protection of the active agent from oxidation or photodegradation by UV radiation

Having described the invention, the following examples are given to illustrate specific applications of the invention. These specific examples are not intended to limit the scope of the invention described in this application.

### Example 1

Entrapped active agent—The entrapment system is formed by addition of the active agent chosen from a list of active agents that are either naturally derived or synthetically derived analogs of the natural products. The halloysite clay is to be substantially tubular with an aspect ratio between 1 and 1000. The active agent is entrapped in the clay by initially mixing it with alphatocopherol, methyl anthranilate, and an isocyanate or a cyanoacrylate. Entrainment is by vacuum removal of water and entrained gasses and the introduction of the agent. Once entrapped the clay is then suspended in a solution of propylene glycol and allowed to crosslink. Then the excess glycol is removed and the clay reserved.

### Example 2

Coating—The coating is then formed by utilization of an acidic solution of chitosan wherein the chitosan concentration varies from 1 to 20% w:v in water, with a surfactant added at the rate of 0.001 and 0.025% w:v in water. Other additives such as pigments, anti-settling agents, added surfactants, crosslinking catalysts, and the entrapped active agent are added to the mixture. The coating is crosslinked utilizing a blocked isocyanate that may cure at ambient temperatures or at slightly elevated temperatures and at a pH between 8-11. If a sulfonate blocked isocyanate is used it may be directly added to the coating at the time of manufacture. If another blocking strategy is utilized then the crosslinker is added within a few minutes of application, but not more than a couple of hours. A suitable ratio of cyanate to hydroxyl or amine functional group is from 0.2:1 to 0.75 to 1. Application is by spray, roller, or brush to clean surfaces.

Obviously, many modifications and variations of the present invention are possible in light of the above teachings. It is therefore to be understood that the claimed invention may be practiced otherwise than as specifically described.

What is claimed is:

1. An antifouling coating comprising:
a polymer made by crosslinking a chitosan with a blocked polyisocyanate;
a halloysite clay in the form of microtubules; and
a biologically active compound entrapped in the microtubules.

2. The coating of claim 1, wherein the blocked polyisocyanate is sulphone blocked hexamethylene diisocyanate.

3. The coating of claim 1, wherein the chitosan and the blocked polyisocyanate are further crosslinked with a polyethylene glycol.

4. The coating of claim 1, wherein the biologically active compound is selected from the group consisting of a 2-furyl-alkylketone, 2-furyl-pentylketone, 2-furyl-heptylketone, 2-furyl-octylketone, and 2-furyl-nonylketone.

5. The coating of claim 1, wherein the microtubules are coated with a polyethylene glycol polyurethane.

6. The coating of claim 1, wherein the coating further comprises:
polydimethylsiloxane.

7. The coating of claim 1, wherein the coating further comprises:
an antioxidant; and
a UV inhibitor.

8. A method comprising:
applying to a surface a mixture comprising:
an aqueous solution of a chitosan;
a blocked polyisocyanate;
a halloysite clay in the form of microtubules; and
a biologically active compound entrapped in the microtubules; and
crosslinking the chitosan with the blocked polyisocyanate.

9. The method of claim 8, wherein the crosslinking comprises further crosslinking with a polyethylene glycol.

10. The method of claim 8, further comprising:
forming a polyethylene glycol polyurethane coating on the tubules.

11. The method of claim 8, wherein the mixture further comprises:
polydimethylsiloxane.

12. The method of claim 8, wherein the mixture further comprises:
an antioxidant; and
a UV inhibitor.
